(19) United States
(12) Patent Application Publication
(10) Pub. No.: US 2021/0236692 A1
(43) Pub. Date: Aug. 5, 2021

(54) REGENERATION OF DISEASES INTERVERTEBRAL DISCS

(71) Applicant: NATIONAL UNIVERSITY OF IRELAND, GALWAY, GALWAY (IE)

(72) Inventors: Abhay PANDIT, Galway (IE); Isma Liza MOHD ISA, Galway (IE)

(73) Assignee: NATIONAL UNIVERSITY OF IRELAND, GALWAY, GALWAY (IE)

(21) Appl. No.: 17/088,234

(22) Filed: Nov. 3, 2020

Related U.S. Application Data
(63) Continuation of application No. 16/339,527, filed on Apr. 4, 2019, now abandoned.

(30) Foreign Application Priority Data
Oct. 4, 2016 (GB) GB 1616849.4

(51) Int. Cl. A61L 27/20; A61L 27/52; A61L 27/54

(52) U.S. Cl. CPC A61L 27/20; A61L 27/52; A61L 27/54; A61L 2430/38; A61L 2300/412; A61L 2300/414; A61L 2400/06; A61L 2300/236

(57) ABSTRACT

A hydrogel composition for use in a method of regeneration of an intervertebral disc, or suppression of discogenic pain, in a mammal with intervertebral disc degeneration is described. The hydrogel composition comprises cross-linked high molecular weight hyaluronan. The hydrogel composition is implanted in the mammal at a site of intervertebral disc degeneration.

### FIELD OF THE INVENTION

The present invention relates to methods for regeneration of diseased intervertebral discs. Also contemplated are methods of treating discogenic pain caused by intervertebral disc degeneration.

### BACKGROUND TO THE INVENTION

Low back pain (LBP) is a common health problem that affects 60-80% of the population of developed countries at some stage in their lives. Patients develop chronic back pain followed by long-term disability leading to morbidity, with severe socio-economic impacts on society. The majority of cases of LBP are caused by intervertebral disc (IVD) degeneration and most patients remain asymptomatic with some experiencing discogenic pain. Current therapy for IVD degeneration focuses on spinal fusion devices such as the Infuse® Bone Graft/LT-Cage® Lumbar Tapered Fusion Device (Medtronic), AccuLIF® Expandable Lumbar Interbody Fusion Technology (Stryker), Anterior Lumbar Interbody Fusion (ALIF) (DePuy), XLIF® (NuVasive), Mobi-C® cervical disc replacement device (Zimmer Biomet Holdings/LDR), CALIBER® (Globus Medical, Inc.), ALPHATEC SOLUS® (Alphatec Holdings), SKYHAWK® Lateral Interbody Fusion System (Orthofix International) which aim to alleviate pain through the removal of a damaged or diseased disc through an anterior or lateral approach. These procedures involve the complete removal of the intervertebral disc and the implantation of an interbody fusion device to restore intervertebral height and fuse the affected vertebral bodies. Spinal fusion devices involve extensive bone work, which leads to more back pain and longer recovery times. The lateral facet joints and transverse processes (specific parts of your vertebra, both located on the sides of each vertebra) are typically exposed during a fusion. This necessitates more muscle dissection. Bone graft or bone substitutes are also placed in the spine to help the patients' bones to gradually fuse together, resulting in inflammation which leads to more scarring (arachnoiditis) and subsequent pain. In addition, a fused segment causes more stress on the level above and below the fusion which is known as adjacent segment disease. A rigid segment next to a mobile segment causes additional stresses at the mobile segment, resulting in degeneration.

Furthermore, most fusions involve placing rods and screws that aim to stabilize the spine until the bony fusion grows solid which causes nerve irritation and new or residual leg pain/weakness. In addition, limitations in mobility are experienced by patients following spinal cord fusion. Critically these technologies are not regenerative in nature resulting in the need for repeated surgery and do not address the underlying disease pathology.

Frith et al (Biomaterials, 2013, 34, No. 37, 9430-9440) discloses the fabrication of a pentosan polysulphate (PPS)—low molecular weight hyaluronan—amine terminated 8-arm polyethylene glycol incorporating mesenchymal precursor cells, for use as a delivery matrix for mesenchymal precursor cells in the treatment of IVD degeneration. The formation of cartilage-like tissue in the treated disc was significantly enhanced by the incorporation of PPS into the hydrogels.

US2016/038643 discloses an implantable hydrogel precursor composition comprising: a cross-linkable polymer matrix, in particular high molecular weight hyaluronic acid, for treatment of cartilage tissue and nerve tissue.

Chen et al. (Acta Biomaterialia, 2013, Vol. 9, No. 2, 5181-5193) discloses in-situ forming hydrogels composed of oxidized high molecular weight hyaluronic acid and gelatin for nucleus pulposus regeneration. An injectable oxidized hyaluronic acid-gelatin-adipic acid dihydrazide (oxi-HAG-ADH) hydrogel was prepared where high molecular weight (1900 kDa) hyaluronic acid was cross-linked with various concentrations of gelatin. Hyaluronic acid is described as the excipient and not the main component of the hydrogel. In addition the therapeutic mode of action is through the encapsulation of nucleus pulposus cells in the hydrogel.

US2010/029789 discloses a hydrogel comprising (a) a first network comprising photocrosslinkable hyaluronan and (b) a second network comprising a hydrophilic polymer or a monomer thereof which is preferably an acrylamide, wherein (a) and (b) are combined and photocrosslinked. The hydrogel is useful as a load bearing orthopaedic implant and/or spinal disc substitute.

US2009//252700 discloses the development of a synthetic nucleus pulposus comprising polycarboxylate, polyamine, or polyhydroxyphenyl macromolecules that have been cross-linked via dihydroxyphenyl linkages and which incorporates mesenchymal stem cells.

US2016/243282 discloses a hydrogel biomaterial comprising a soluble elastin, collagen, and at least one glycosaminoglycan, wherein the collagen and the glycosaminoglycan are crosslinked to one another, the soluble elastin being non-fibrous, and in the form of microspheres within the hydrogel biomaterial comprising nucleus pulposus cells, stem cells, autologous cells and cell growth factors and/or cell differentiation factors.

Pereira et al (Biomaterials, 2014, Vol. 35, 8144-8153) discloses a thermoreversible hyaluronan-poly(N-isopropylacrylamide (HAP) hydrogel containing stromal cell derived factor-I (SDF-1) as a chemoattractant delivery system to recruit human mesenchymal stem cells (MSCs) in degenerative intervertebral discs.

Isa et al. (Biomacromolecules 2015, 16, 1714-1725) discloses the use of cross-linked high molecular weight HA hydrogels to modulate the inflammatory receptor of IL-1R1, MyD88 and neurotrophin expression of nerve growth factor (NGF) and brain-derived neurotrophic factor (BDNF) in an in vitro inflammation model of nucleus pulposus (NP) in-vitro.

It is an object of the invention to overcome at least one of the above-referenced problems.

### SUMMARY OF THE INVENTION

Previous studies of intervertebral disc degeneration by needle puncture in rats have indicated a clear tendency to progressive disc degeneration over a 30-day period after injury (Issy et al, Braz. J. Med. Bio. Res. 2013 March; 46(3)). The Applicant has discovered that progressive disc degeneration in this animal model can be arrested, and even reversed, by treating the injured disc with a high molecular weight hyaluronan hydrogel implant (FIGS. 1 and 3). The Applicant has also discovered that a high molecular weight hyaluronan hydrogel modulates the proteome signature of cells in both the nucleus polposus (NP) and annulus fibrosus (AF) in the same model of IVD degeneration to promote disc regeneration by the formation of functional extracellular matrix (ECM) (FIGS. 2A-2D). The Applicant has also discovered that a high molecular weight HA implant can inhibit pain sensitization in peripheral regions of the disc and suppress pain processes in a rat tail model of IVD by attenuation of hyperalgesia, nociception, hyperinnervation and/or hypoalgesia. (FIGS. 4-9G).

According to a first aspect of the present invention, there is provided a hydrogel composition comprising of high molecular weight hyaluronan for use in a method of regeneration of an intervertebral disc or suppression of discogenic pain, in a mammal. Generally, the mammal is afflicted with intervertebral disc (IVD) disease, for example IVD degeneration. Typically, the hydrogel composition is implanted in the mammal at a site of intervertebral disc disease or degeneration. Suitably, the hyaluronan is crosslinked.

In one embodiment, the therapy causes no decrease in the height of the treated disc compared with an untreated disc after a treatment period of 56 days.

In one embodiment, the therapy causes an increase in the height of the treated disc compared with an untreated disc after a treatment period of 56 days.

In one embodiment, the use of the invention also includes its use in reducing discogenic pain associated with IVD disease, especially IVD degeneration.

In one embodiment, the mammal afflicted with IVD disease has a herniated disc.

In a related aspect, the invention relates to a hydrogel composition comprising high molecular weight hyaluronan for use in a method of suppressing discogenic pain in a mammal, typically a mammal suffering from IVD disease, especially IVD degeneration.

In a related aspect, the invention relates to a hydrogel composition comprising high molecular weight hyaluronan for use as an analgesic.

In a related aspect, the invention relates to a hydrogel composition comprising high molecular weight hyaluronan for use in a method of suppressing pain at a locus in a mammal, in which the hydrogel composition is typically implanted at the locus. In one embodiment, the hydrogel reverses thermal hyperalgesia, mechanical hyperalgesia and/or hypoalgesia at the locus to alleviate pain.

In a related aspect, the invention relates to a hydrogel composition comprising high molecular weight hyaluronan for use in a method of attenuating hyperalgesia or hypoalgesia at a locus in a mammal, in which the hydrogel composition is typically implanted at the locus. In one embodiment, the hydrogel composition reverses hyperalgesia or hypoalgesia. In one embodiment, the hyperalgesia is selected from thermal hyperalgesia and mechanical hyperalgesia.

In a related aspect, the invention relates to a hydrogel composition comprising high molecular weight hyaluronan for use as an anti-inflammatory agent in which the hydrogel composition typically attenuates systemic pro-inflammatory cytokines, to alleviate pain.

In a related aspect, the invention relates to a hydrogel composition comprising high molecular weight hyaluronan for use in a method of treating or preventing an inflammatory disorder in a mammal. In one embodiment, the hydrogel composition typically attenuates the level or expression of systemic pro-inflammatory cytokines. In one embodiment, the inflammatory disorder is characterised by elevated levels of systemic pro-inflammatory cytokines.

In a related aspect, the invention relates to a hydrogel composition comprising high molecular weight hyaluronan for use in a method of suppressing pain in a mammal, especially a mammal suffering from a joint disease. The joint disease may be a degenerative condition or a condition caused by trauma, or both. In one embodiment, the joint disease comprises articular cartilage damage. In one embodiment, the joint disease is arthritis, for example rheumatoid arthritis.

In one embodiment, the HA hydrogel is cross-linked. Use of different cross-linking agents in the hydrogel matrix provides for a composition having a tailored HA degradation profile, and allows the use of different crosslinking agents to provide for a tunable HA hydrogel scaffold. In one embodiment, the hydrogel is crosslinked in-situ at a locus in the body. This can be achieved by using a dual syringe (i.e. a duploject system) that keeps the hydrogel and crosslinking agent separate prior to injection and mixes them as they are ejected from the syringe.

In one embodiment, the HA is chemically cross-linked. In one embodiment, the crosslinking moiety (agent) is a functionalised PEG, for example PEG-amine. In one embodiment, cross-linking initiation is performed with EDC/NHS or 4-(4,6-dimethoxy-1,3,5-triazin-2-yl)-4-methylmorpholinium chloride (DMTMM) chemistry. Other methods of cross-linking include thermal cross-linking. In one embodiment, the ratio of cross-linking agent to HA is 1:1 to 1:10 (by weight), typically 1:1 to 1:5, and preferably about 1:1 to 1:3. In one embodiment, the ratio of the cross-linking agent to HA is about 1:2 (by weight).

In one embodiment, the hyaluronan (HA) is positively charged. This can be achieved by derivatizing the HA with a moiety that imparts a net positive charge on the HA molecule (for example a cation). Examples of moieties that can be employed to derivatize HA include aminopropyl imidazole. In this specification, the term HA includes both derivatized and non-derivatized HA. Method of producing positively charged HA, for example cationized HA, are described in the literature and include carboxyl and hydroxyl group modification using quaternary ammonium containing groups (US2009/0281056 and US2010/0197904).

In one embodiment, the composition comprises a therapeutically effective amount of HA. In one embodiment, the composition comprises about 0.1% to about 10% HA (weight %). In one embodiment, the composition comprises about 0.5% to about 5% HA (weight %). In one embodiment, the composition comprises about 0.1% to about 1% HA (weight %). In one embodiment, the composition comprises about 1.0% to about 10% HA (weight %). In one embodiment, the composition comprises about 0.5% to about 2% HA (weight %). In one embodiment, the composition comprises about 5.0% to about 10% HA (weight %).

In one embodiment, the hydrogel composition is a hydrogel, optionally combined with additional components, for example pharmaceutically or biologically active agents such as cells, drugs, or HA particles. In one embodiment, the hyaluronan in the hydrogel is a hyaluronan homopolymer. In one embodiment, the hydrogel comprises a single polymer network. In one embodiment, the HA binds to cell surface receptors, including CD44.

In one embodiment, the method of treatment comprises administering the composition periodically during a treatment period. The frequency of administration depends on a number of factors including the status of the disease, the age of the patient, and the effectiveness of the treatment. In one embodiment, the composition is administered once weekly, monthly, six monthly, or yearly. In one embodiment, the composition is administered twice monthly. In one embodiment, the composition is administered once monthly. In one embodiment, the composition or particle is administered between 1 and 10 times during the treatment period. In one embodiment, the treatment period is between 1 week and 6 months.

In one embodiment, pain (for example discogenic or joint pain) is suppressed by inhibiting sensory hyper-innervation and nociception, and/or attenuating systemic pro-inflammatory cytokines, to alleviate pain.

In one embodiment, the hydrogel composition down-regulates molecular markers of nociception; typically substance P and/or c-Fos to alleviate pain.

In one embodiment, the hydrogel composition attenuates (for example reverses) thermal hyperalgesia, mechanical hyperalgesia and/or hypoalgesia to alleviate pain.

In one embodiment, the hydrogel composition down regulates circulating pro-inflammatory cytokines to attenuate the injury or trauma induced systemic inflammatory response.

In one embodiment, the hydrogel composition modulates endogenous extracellular matrix production to maintain or increase disc height.

In one embodiment, the hydrogel composition modulates the glycosolation profile of the intervertebral disc.

In one embodiment, the hydrogel composition of the invention comprises high molecular weight hyaluronan particles.

In one embodiment, the HA particles are nano-sized particles, typically having an average particle size of 100-900 nm. In one embodiment, the HA particles have an average size of 300 to 700 nm. In one embodiment, the HA particles have an average size of 400 to 600 nm. In one embodiment, the HA particles are agglomerates of nano-sized HA particles, which agglomerates may have an average dimension of 500 nm to 10 microns.

The compositions/hydrogels of the invention may include additional components. Thus, the HA particles may comprise one or more additional components. The carrier phase (i.e. hydrogel) may incorporate one or more additional components. Both the HA particles and the carrier phase may, independently, incorporate one or more additional components. The component may be a pharmaceutically or biologically active agent. The component may be a cell, cell component, polysaccharide, protein, peptide, polypeptide, antigen, antibody (monoclonal or polyclonal), antibody fragment (for example an Fc region, a Fab region, a single domain antibody such as a nanobody or VHV fragment), a conjugate of an antibody (or antibody fragment) and a binding partner such as a protein or peptide, a nucleic acid (including genes, gene constructs, DNA sequence, RNA sequence, miRNA, shRNA, siRNA, anti-sense nucleic acid). The component may be a cellular product such as a growth factor (i.e. EGF, HGF, IGF-1, IGF-2, FGF, GDNF, TGF-alpha, TGF-beta, TNF-alpha, VEGF, PDGF and an interleukin. The component may be a drug, for example, a drug to relieve pain such as non-steroidal anti-inflammatory drug (such as Ibuprofen, Ketoprofen or Naproxen), aspirin, acetaminophen, codeine, hydrocodone, an anti-inflammatory agent such as a steroidal anti-inflammatory agent, an anti-depressant, an anti-histamine, or an analgesic. The cell may be autologous, allogenic, xenogenic. The cell may be a stem cell. The stem cell may be selected from the group comprising a side population, endothelial, hematopoietic, myoblast, placental, cord-blood, adipocyte and mesenchymal stem cells. The cells may be engineered to express a biological product, for example a therapeutic biological product such as a growth factor.

In one embodiment, a weight ratio of HA particles to HA hydrogel matrix is about 1:9 to 9:1. In one embodiment, the weight ratio of HA particles to HA hydrogel matrix is about 1:5 to 5:1. In one embodiment, the weight ratio of HA particles to HA hydrogel matrix is about 1:4 to 4:1. In one embodiment, the weight ratio of HA particles to HA hydrogel matrix is about 1:3 to 3:1. In one embodiment, the weight ratio of HA particles to HA hydrogel matrix is about 1:2 to 2:1. In one embodiment, the weight ratio of HA particles to HA hydrogel matrix is about 1:1. In one embodiment, the HA particles are suspended in the HA gel.

In one embodiment, the HA particles are cross-linked with a cross-linking moiety. In one embodiment, the HA particles are chemically cross-linked. In one embodiment, the cross-linking moiety (agent) is a functionalised PEG, for example PEG-amine. In one embodiment, cross-linking initiation is performed with EDC/NHS or 4-(4,6-dimethoxy-1,3,5-triazin-2-yl)-4-methylmorpholinium chloride (DMTMM) chemistry. Other methods of crosslinking include thermal crosslinking. In one embodiment, the ratio of cross-linking agent to HA is 1:1 to 1:10 (by weight); typically 1:1 to 1:5, and preferably about 1:1 to 1:3. In one embodiment, the ratio of cross-linking agent to HA is about 1:2 (by weight). Methods of crosslinking HMW HA with PEG-amine are described below and in Ise et al (Biomacromolecules 2015, 16, 1714-1725).

In one embodiment, the cross-linking moiety of the HA particles are different to the cross-linking moiety of the HA hydrogel matrix. Use of different cross-linking agents in the particles and hydrogel matrix provides for a composition having a tailored HA degradation profile, and allows the use of different cross-linking agents to provide for a tunable HA hydrogel scaffold.

Other aspects and preferred embodiments of the invention are defined and described in the other claims set out below.

### BRIEF DESCRIPTION OF THE FIGURES

FIG. 1: Disc height changes in the rat tail disc injury model: Bar graph represent the % disc height changes between sham, injured and injured HA treated groups over 7 and 28 days. Mean±SD. *p<0.05 vs injury.

FIGS. 2A-2D: Proteomic analysis of intervertebral disc by mass-spectometry. (FIG. 2A) Venn diagram (left) showing the distribution of total proteins in annulus fibrosus and nucleus pulposus tissue associated with post-implantation HA hydrogel. (FIG. 2B) Heat map represented proteome signature of AF and NP tissue in sham, injury and implantation HA hydrogel. (FIG. 2C) Modulation of extracellular matrix deposition in NP. (FIG. 2D) Modulation of extracellular matrix deposition in AF. Protein expression was presented as log 2 (ratio) using PEAKS, false discovery rate≤1% of 1 unit peptide, N=5).

FIG. 3: Disc height changes in the rat tail disc injury model: Bar graph represent the % disc height changes between sham, injured and injured HA treated groups over 7, 28 and 56 days. Mean±SD. *p<0.05 vs injury.

FIG. 4: Overview of the Experimental design of the HA hydrogel implanted subcutaneously in the rat tail model before pain behaviour assessment at pre- and post-injury.

FIGS. 5A-5B: Implantation of the HA hydrogel displayed increase latency time in Hargreaves test until day 29 and this suggest HA alleviates thermal hyperalgesia as comparable to morphine treatment as a positive control. *p<0.05, one-way ANOVA repeated measure, N=10-20. Data presented as the mean±standard error mean.

FIGS. 6A-6F: HA hydrogel alleviated the pain phenotype following puncture-induced IVD injury. (FIG. 6A) Hargreaves test reduced latency time in the treatment group compared to sham.

(FIG. 6B and FIG. 6C) Implantation of HA hydrogel reduced mechanical allodynia until post-operative day 29.

(FIG. 6D) Tail flick test demonstrated lower latency time in the implantation group of animals. (FIG. 6E and FIG. 6F) Molecular markers of nociception, substance P and c-Fos were down-regulated upon implantation HA hydrogel respectively. *Significant differences were noted between the different groups (n=10-20 one-way ANOVA repeated measure p<0.05). Data presented as the mean±standard error mean.

FIGS. 7A-7B: HA Hydrogel attenuated peripheral sensitization in AF and NP tissue. (FIG. 7A) Confocal microphotographs indicated evidence that nerve ingrowth stained by GAP43 with yellow label, sensory neuropeptide using CGRP antibody with purple label, and nociceptor of ion channel TRPV1 with green label exhibited reduced fluorescence following implantation of the HA hydrogel. (FIG. 7B) Quantification of the volume fraction of GAP43, CGRP and TRPV1 which were reduced in AF and NP tissue following implantation of the HA hydrogel. *Significant differences were noted between the different groups (n=3-5 one-way ANOVA p<0.05). Data presented as the mean±standard error mean. Scale bar=50 μm.

FIGS. 8A-8E: Regulation of circulating inflammatory cytokines associated pain upon implantation: (FIG. 8A-8D) HA hydrogel decreased systemic inflammation by down regulating circulating the pro-inflammatory cytokines (IL-6, IL-1β, IFN-Y and TNF-α). (FIG. 8E) An increase of the anti-inflammatory marker (IL-10) was observed in the blood plasma. *Significant differences were noted between the different groups (n=3-5 one-way ANOVA p<0.05). Data presented as the mean±standard error mean.

FIGS. 9A-9G: Glycosignature in response to implantation of the HA hydrogel in a novel rat model of pain associated with IVD injury. (FIG. 9A) Confocal images show yellow fluorescence of keratan sulfate presented intracellularly and purple label of chondroitin sulfate in extracellular matrix (FIG. 9B) Quantification volume fraction of positive stained chondroitin sulfate in AF and NP tissue was maintained as similar as sham, however keratan sulfate reduced after implantation HA hydrogel. (FIG. 9C and FIG. 9D) An iterative increase of GSIB4 binding to α-galactose was observed upon injury and decreased in response to HA hydrogel. (FIG. 9E) α2á6 sialylated N-acetylgalactosamine motifs detected by SNA-I in healthy, injured and post-implantation HA hydrogel in NP and AF tissues at 28 days. (FIG. 9F) sialylated N-acetyl-D-glucosamine motifs detected by WGA in healthy, injured and post-implantation HA hydrogel in NP and AF tissues at 28 days. (FIG. 9G) Clustering analysis of the average glycosylation profile post-implantation HA hydrogel. *Significant differences were noted between the different groups (n=3-5 one-way ANOVA p<0.05). Data presented as the mean±standard error mean. Scale bar=50 μm.

FIG. 10: NMR-H for cross linked particles using as coupling reagent: (a) EDC/NHS, (b) DMTMM (4-(4,6-Dimethoxy-1,3,5-triazin-2-yl)-4-methylmorpholine).

FIG. 11: NMR-H of final products obtained under three different reaction conditions after centrifugation at 1500 rpm. Purple: control reaction, coupling reagent was not used; Red: EDC was used as coupling reagent; Green: DMTMM was used as coupling reagent.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reversal of hyperalgesia in an intervertebral disc). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

As used herein, the term “regeneration” as applied to an intervertebral disc means inhibition of disc degeneration, slowing or attenuating the rate of disc degeneration, or more preferably, reversal of disc degeneration where degeneration is completely inhibited and regeneration of the disc occurs. Inhibition and reversal of disc degeneration can be measured in a subject using various methods, including the rat tail model described herein.

As used herein, the term “intervertebral disc disease” refers to damaged intervertebral disc tissue caused by trauma or a disc degenerative condition. Disc degeneration can occur naturally, often associated with ageing, or can manifest in many clinical conditions including spinal stenosis and instability, radiculopathy, myelopathy and disc herniation. Disc degeneration is generally but not always associated with back pain.

As used herein, the term “intravertebral disc degeneration” or “IVD degeneration” refers to a pathological process generally involving degeneration over time of IVD's and usually characterised by one or more of degeneration of fibrocartilage, splits in the annulus fibrosus which leads to herniation of elements of the nucleus pulposus, shrinkage of the nucleus pulposus. Inflammation is often involved, and disc degeneration often leads to lower back pain.

As used herein, the term “implanted” as applied to administration of the hydrogel refers to implanting the hydrogel in the body at the locus of the disc disease or degeneration. The hydrogel may be implanted around the circumference of the disc, or implanted in the spine between an affected disc and an adjacent vertebra. In one embodiment, the hydrogel is implanted into the lumbar associated musculature adjacent the affected disc (for example, the psoas major muscle, multifidus muscle, the transversospinalis muscle or the sacrospinalis muscle). The hydrogel may be implanted by any suitable means, for example by injection using a suitable syringe or by surgical implantation. A hydrogel for surgical implantation generally has a higher viscosity that a hydrogel for injection. In one embodiment, the hydrogel and crosslinker are injected separately to allow in-situ crosslinking of the hydrogel at the target site using, for example, a duploject injection system.

As used herein, the term “hyaluronan” or “hyanuronic acid” or “HA” refers to the anionic non-sulphated glycosaminoglycan that forms part of the extracellular matrix in humans and consists of a repeating disaccharide→4)-β-d-GlcpA-(1→3)-β-d-GlcpNAc-(1→. Hyaluronan is the conjugate base of hyaluronic acid, however the two terms are used interchangeably and both may be employed in the present invention. When a salt of hyaluronic acid is employed, the sale is generally a sodium salt, although the salt may be employed such a calcium or potassium salts. The hyaluronic acid or hyaluronan may be obtained from any source, including bacterial sources. Hyaluronic acid sodium salt from Streptococcus equi is sold by Sigma-Aldrich under the product reference 53747-1G and 53747-10G. Microbial production of hyaluronic acid is described in Liu et al (Microb Cell Fact. 2011; 10:99). The term also includes derivatives of HA, for example HA derivatised with cationic groups as disclosed in US2009/0281056 and US2010/0197904, and other types of functionalised derivatives, such as the derivatives disclosed in Menaa et al (J. Biotechnol Biomaterial S3:001 (2011)), Schante et al (Carbohydrate Polymers 85 (2011)), EP0138572, EP0216453, EP1095064, EP0702699, EP0341745, EP1313772 and EP1339753.

As used herein, the term “hyaluronan hydrogel” or “hyaluronan hydrogel matrix” means a three-dimensional network of hyaluronan polymers in a water dispersion medium. In one embodiment, the hyaluronan polymers are crosslinked to form the three-dimensional network. Typically, the hydrogel matrix is formed of hyaluronan homopolymer, and not a hyaluronan containing copolymer. In one embodiment, the hydrogel is injectable (i.e. has a viscosity that allows the delivery of the hydrogel in-vivo by injection. In one embodiment, the hydrogel is suitable for implantation. Methods of generating HMW HA hydrogels are described below and in Ise et al (Biomacromolecules 2015, 16, 1714-1725).

As used herein, the term “high molecular weight” as applied to hyaluronic acid typically means a molecular weight of greater than 500 KDa. In one embodiment, the high molecular weight has a molecular weight of greater than 600 KDa. In one embodiment, the high molecular weight has a molecular weight of greater than 700 KDa. In one embodiment, the high molecular weight has a molecular weight of greater than 800 KDa. In one embodiment, the high molecular weight has a molecular weight of greater than 900 KDa. In one embodiment, the high molecular weight has a molecular weight of greater than 1000 KDa. In one embodiment, the high molecular weight has a molecular weight of greater than 1100 KDa. In one embodiment, the high molecular weight hyaluronan has a molecular weight of between 500 and 5000 KDa. In one embodiment, the high molecular weight hyaluronan has a molecular weight of between 500 and 2000 KDa. In one embodiment, the high molecular weight hyaluronan has a molecular weight of between 500 and 1500 KDa. In one embodiment, the high molecular weight hyaluronan has a molecular weight of between 500 and 1000 KDa

As used herein, the term “cross-linked” as applied to hyaluronic acid means that hyaluronic acid polymer chains are covalently cross-linked with a crosslinking agent to form a three-dimensional network. Cross-linked HA hydrogels are described in the literature, for example in Kenne et al (Carbohydrate Polymers, Vol. 91, Issue 1 (2011)), Segura et al (Biomaterials, Vol. 26, Issue 4 (2005)), Yeom et al (Bioconjugate Chem, Vol. 21(2) 2010), U.S. Pat. Nos. 8,124,120, and 6,013,679. The term “cross-linking agent” generally means a molecule containing two or more functional groups that can react with HA. Examples of cross-linking agents include ethylene glycol crosslinking agents, including functionalised polyethylene glycol (PEG), for example PEG-amine and PEG diglycidylether (EX810), 1-ethyl-3-(3-dimethylaminopropyl) carboimide (EDC), divinyl sulfone (DVS) and ethylene glycol diacrylates and dimethacrylates, derivatives of methylenebisacrylamide (Sigma-Aldrich). Methods of crosslinking HMW HA with PEG-amine are described below and in Isa et al. (Biomacromolecules 2015, 16, 1714-1725). The hydrogel of the invention may be crosslinked. The hydrogel may be crosslinked prior to administration, or it may be crosslinked during or after administration. For example, the hydrogel and crosslinking agent may be administered using a syringe that keeps the two components separate until delivery where the components are mixed to allow in-situ crosslinking of the hydrogel. This may be achieved using a Duploject injection system.

As used herein, the term “nano-sized” as applied to hyaluronan particles means having an average dimension in the nanometer range. For example, the HA particles may have an average size of 1 to 1000 nm, typically 100 to 900 nm, typically 200 to 800 nm, preferably 300 to 700 nm, and more preferably 400 to 600 nm. In one embodiment, the HA particles have an average size of 500+/−100 nm. Particle size is measured using a Malvern Zetasizer (nano range).

As used herein, the term “discogenic pain” refers to pain generating from damaged intervertebral discs. These results from the release of neurotrophins (including beta-nerve growth factor (β-NGF) and brain-derived neurotrophic factor (BDNF)) and neuropeptides (including calcitonin gene-related peptide (CGRP) and substance P) which promote the innervation of peptidergic small neurons into the aneural discs and induce sensitization of pro-nociceptors (TRPV1 and Trk A) for pain transmission.

As used herein, the term “nociception” generally refers to a perception or sensation of pain where the sensory nervous system's responds to potentially harmful stimuli. A nociceptor is a receptor at the end of a sensory nerve fibre that responds to harmful/painful stimuli by sending danger signals to the brain.

As used herein, the term “hyperalgesia” generally refers to an increased sensitivity to pain, which is caused by sensitization of nociceptors or damage to peripheral nerves. The HA hydrogel of the invention alleviates thermal hyperalgesia around the injury site.

As used herein, the term “hypoalgesia” (or “hypalgesia”) denotes a decreased sensitivity to painful stimuli. When a painful stimulus is applied outside the receptive field which is distal to the site of injury, it can lead to a to a phenomenon where pain inhibits pain. The alteration of this endogenous pain modulation is clinically presented as hypoalgesia. The HA hydrogel treatment suppresses a hypoalgesia phenomenon when a painful stimulus applied far away (distal) from the site of injury.

As used herein, the term “hyperinnervation” refers to an increased sensitivity to pain, which is caused by damage to nociceptors or peripheral nerves. Hyperinnervation involves increased innervation of nerve fibres (nerve ingrowth) into the tissue. Sensory hyperinnervation refers to increased distribution or population of sensory nerve fibres into the disc tissue. Increased nerve ingrowth is defined as hyperinnervation. The implanted HA-hydrogel inhibits injury-induced peripheral sensory innervation in the disc.

As used herein, the term “Inflammatory disorder” means an immune-mediated inflammatory condition that affects mammals especially humans and is generally characterised by dysregulated expression of one or more cytokines. Examples of inflammatory disorders include skin inflammatory disorders, inflammatory disorders of the joints, inflammatory disorders of the vertebrae and/or vertebral discs, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the intervertebral discs include intervertebral disc degeneration. Examples of inflammatory disorders of the cardiovascular system are cardiovascular disease and atherosclerosis. Examples of autoimmune diseases include Type 1 diabetes, Graves disease, Guillain-Barre disease, Lupus, Psoriatic arthritis, and Ulcerative colitis. Examples of lung and airway inflammatory disorders include asthma, cystic fibrosis, COPD, emphysema, and acute respiratory distress syndrome. Examples of intestinal inflammatory disorders include colitis and inflammatory bowel disease. Other inflammatory disorders include cancer, hay fever, periodontitis, allergies, hypersensitivity, ischemia, depression, systemic diseases, post infection inflammation and bronchitis. In this specification, the term “Metabolic disorder” should be understood to include pre-diabetes, diabetes; Type-1 diabetes; Type-2 diabetes; metabolic syndrome; obesity; diabetic dyslipidemia; hyperlipidemia; hypertension; hypertriglyceridemia; hyperfattyacidemia; hypercholerterolemia; hyperinsulinemia, and MODY.

As used herein, the term “locus in the body” refers to a specific location within the body, for example a joint, an intervertebral disc, a vertebra, a bone, a tooth, a limb or part of a limb, or an organ such as a bladder, heart, vascular system, liver, kidney, and brain.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

### Protocol for Non-Cross Linked, Cross-Linked HA Hydrogels

Material Specifications:

Hyaluronic acid: High molecular weight (HM Wt.) sodium hyaluronate 1 M. Da (Lifecore Biomedical, USA). CAS No.: 9067-32-7.

PEG-amine: Mw 2000 Da purchased from JenKem Technology USA (Allen, Tex.). CAS No.: 25322-68-3, purity>95%.

N-(3-Dimethylaminopropyl)-N′-ethylcarbodiimide hydrochloride (EDC) (Sigma-Aldrich, USA) CAS Number 25952-53-8, purity˜100%.

N-hydroxysuccinimide (NHS): (Sigma-Aldrich USA). CAS Number 6066-82-6, purity 98%.

Phosphate buffered saline (Sigma-Aldrich, USA) CAS Number P4417-50TAB (pH adjusted to 6.5)

Formulation or Compounding Procedure for Non-Cross Linked Gels:

1. Prepare Phosphate buffered saline by dissolving 1 tablet in 200 ml distilled water, and adjust the pH to 6.5 (store aside).

2. Slowly add required quantities of Hyaluronic acid sodium salt (3 mg/ml, 9 mg/ml, 15 mg/ml) in Phosphate buffered saline at ≤25° C.

3. Stir the solutions over-night on a magnetic stirrer at ≤25° C. Check the pH if it is within the limit 6.5-7.5.

4. Store the hydrogels at cold room conditions (4° C.) for further use.

Formulation or Compounding Procedure for Cross Linked Gels:

1. Slowly add Hyaluronic acid sodium salt at 9 mg/ml in 0.1 M MES buffer (pH 6.0).

2. To the above solution add EDC (4.5 mg) and NHS (2.7 mg) (with respect to each monomer of HA repeating units) in MES buffer and stirred for 15 minutes. Continue mixing, add required quantity of PEG amine (1:1.32 ratio with respect to HA) for cross-linking and the reaction was stirred overnight at ≤25° C.

3. After completion, the reaction mixture was dialyzed for 24-48 h against distilled water using 6000-8000 MW dialysis membrane to remove any unreacted starting materials and salts.

After dialysis the samples were freeze dried and lyophilized to obtain pure cross-linked HA hydrogels. Redisperse the lyophilized powder in PBS to get same concentration of the gels

### Example 2

### Protocol for Crosslinking of HA

Material Specifications

Hyaluronic acid: High molecular weight (HMwt) sodium hyaluronate 1.2×106 Da (Lifecore Biomedical, USA). CAS No.: 9067-32-7.

PEG-amine: Mw 2000 Da purchased from JenKem Technology USA (Allen, Tex.). CAS No.: 25322-68-3, purity>95%

N-(3-Dimethylaminopropyl)-N′-ethylcarbodiimide hydrochloride (EDC) (Sigma-Aldrich USA) CAS Number 25952-53-8, purity˜100%

N-hydroxysuccinimide (NHS): (Sigma-Aldrich USA). CAS Number 6066-82-6, purity 98%

Solvents: 20 wt % sodium sulphate solution in distilled water and 0.1 M MES (2-(N-morpholino)ethanesulfonic acid) buffer

Evaluation of Synthetic Protocol for Cross-Linking of HA (EDC/NHS)

1. 10 mg/mL conc. HA was dissolved in 0.1 M MES buffer for 2 h at room temperature

a. MES buffer facilitates rapid dissolution of HA to obtain a homogeneous solution

b. MES buffer (pH˜6) also facilitates ionization of the carboxylic groups of HA (pKa˜3-4)

2. A solution of 20 wt % Na2SO4, a neutral ionic salt was then added to further induce ionisation of HA

3. To this solution were added solutions of EDC (46 mg, 0.2 M) and NHS (27 mg, 0.2 M) in MES buffer and stirred for 15 minutes

a. MES buffer has been reported in literature to increase the cross-linking efficiency and the yield

4. PEG (5 mg, 1:2 ratio wrt. HA) was then added for cross-linking and the reaction was stirred overnight at RT

5. After completion the reaction mixture was dialyzed for 48 h against distilled water using a 6000-8000 MW dialysis membrane to remove any unreacted starting materials and salts for 48 h

6. After dialysis the samples were freeze dried and lyophilized to obtain pure cross-linked HA particles

7. Characterization by Zeta Potential and SEM

a. An increase in the size and a decrease in the zeta potential was observed and is attributed to the increased cross-linking efficiency as this reduces the number of free carboxylic groups and also the length of the particles

8. Zeta Potential: −24.8 (Mean), 2.17 (Std Dev), −21.6 (max), −27.3 (min)

Evaluation of Synthetic Protocol for Cross-Linking of HA (DMTMM)

1. 500 mg of HA HA was dissolved in 0.1 M MES buffer for 2 h at room temperature

a. MES buffer facilitates rapid dissolution of HA to obtain a homogeneous solution

b. MES buffer (pH˜6) also facilitates ionization of the carboxylic groups of HA (pKa˜3-4)

2. A solution of 20 wt % Na2SO4, a neutral ionic salt was then added to further induce ionisation of HA

3. To this solution add a solution of DMTMM (385 mg)

a. MES buffer has been reported in literature to increase the cross-linking efficiency and the yield

4. PEG (250 mg) was then added for cross-linking and the reaction was stirred overnight at RT

5. DMTMM and Peg-amine were dissolved in 0.1 M MES Buffer to make a total final reaction volume of 125 mL.

6. The reaction was enriched with 8 vol % saturated sodium chloride.

7. The product was precipitated using 96% ethanol (10 mL)

8. The reaction was allowed to stir for 30 mins to allow complete precipitation

9. Centrifuged at 1500 rpm for 10 mins to collect product

10. Several wash steps were subsequently performed and then the product was filtered and kept under vacuum for 48 h

11. The samples were characterization by NMR for purity, TNBSA for degree of cross-linking and SEM for morphological evaluation.

### Example 3

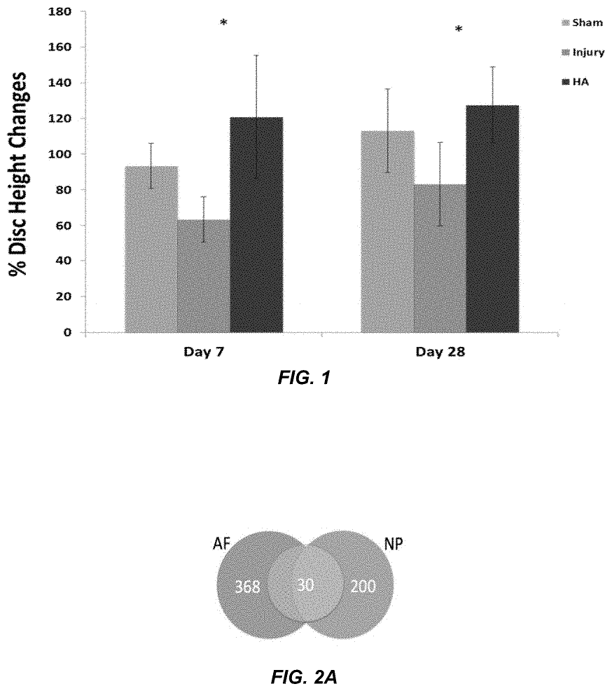

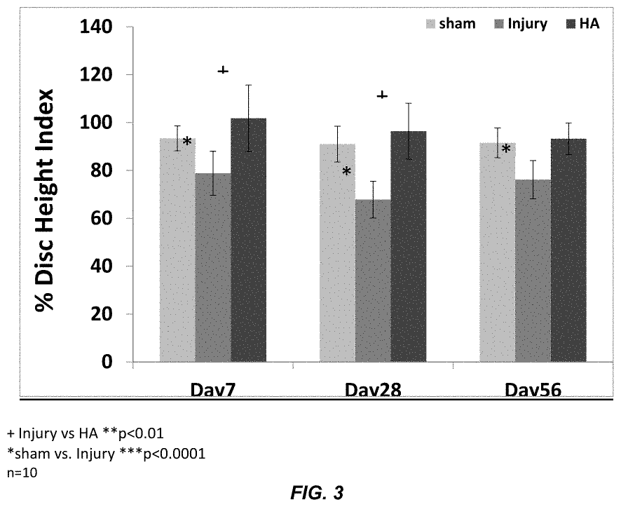

### Disc Height Data (FIGS. 1 and 3)

An in vivo study was conducted in rat-tail model to check the anti-inflammatory effect of hyaluronic acid (HA) microgel. Animals used in this study were 250-350 g of 12 weeks old female rat Sprague Dawley. The experimental design consisted of sham, injury and injury+implantable HA hydrogel groups that have been done on each rat at disc level C4-C5, C5-C6, and Disc C6-C7. The injury was performed by excising out 1×1×1 mm (width, height and depth) of tissue using blade. Then, after inducing the injury, the discs were left as it is or implanted with HA hydrogel depending on the experimental group. The wound was closed in layers using non-absorbable suture, first by suturing the connective tissue layers and then the skin, thereby covering the disc. Analgesics post-surgically and for the next few days (usually for 72 hours) with buprenorphine hydrochloride 0.025 mg/kg every 12 hours, if needed more frequent. After 28 days post-operative, the rats were scarified to harvest disc tissue for immunohistochemistry analysis. The disc height was measured at day 0, 7, 28 and 56. It demonstrates that HA maintains disc height up to 28 days without any significance loss (FIGS. 1 and 3).

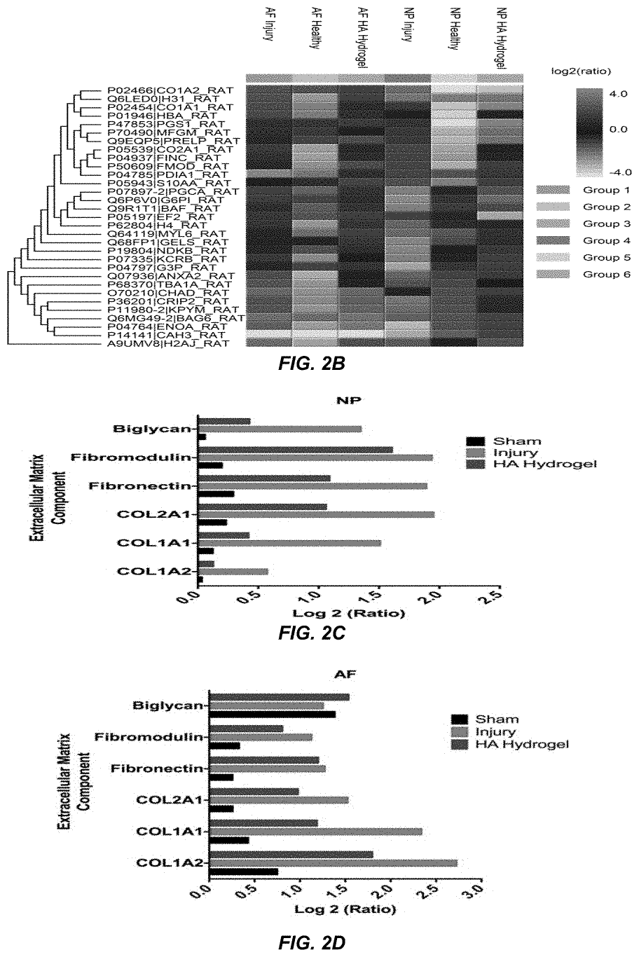

HA Hydrogel Modulates Proteome Signature at Cellular and Extracellular Matrix (FIGS. 2A-2D)

To further obtain mechanistic insight on protein involves in cellular and extracellular signaling pathway, we adopted mass-spectrometry for protein identification and quantification as well as pathway analysis. Based on free label LCMS/MS proteomic analysis, a total of 200 and 386 proteins were identified in ECM AF and NP respectively, associated with the post-implantation HA hydrogel condition. From the heat map analysis, ECM molecules were abundantly detected in both AF and NP tissue (FIG. 2A). Collagen II was up-regulated in both injury and HA hydrogel group.

### Example 4

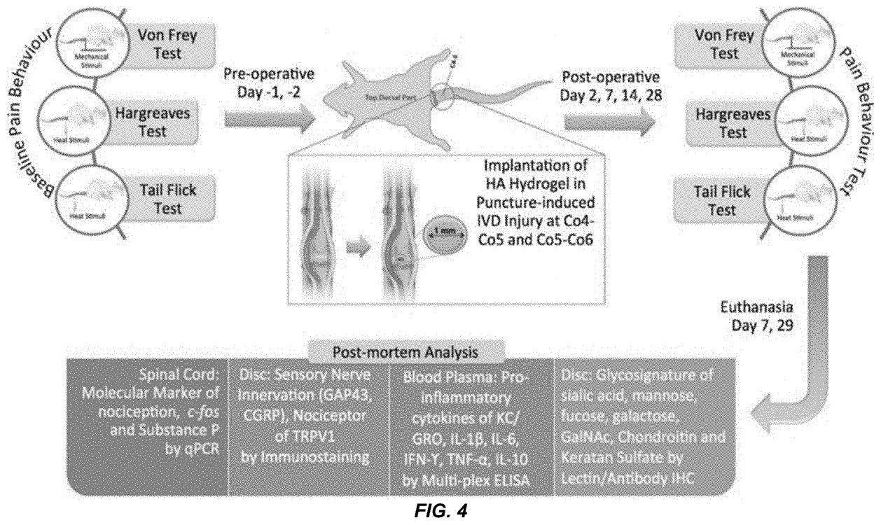

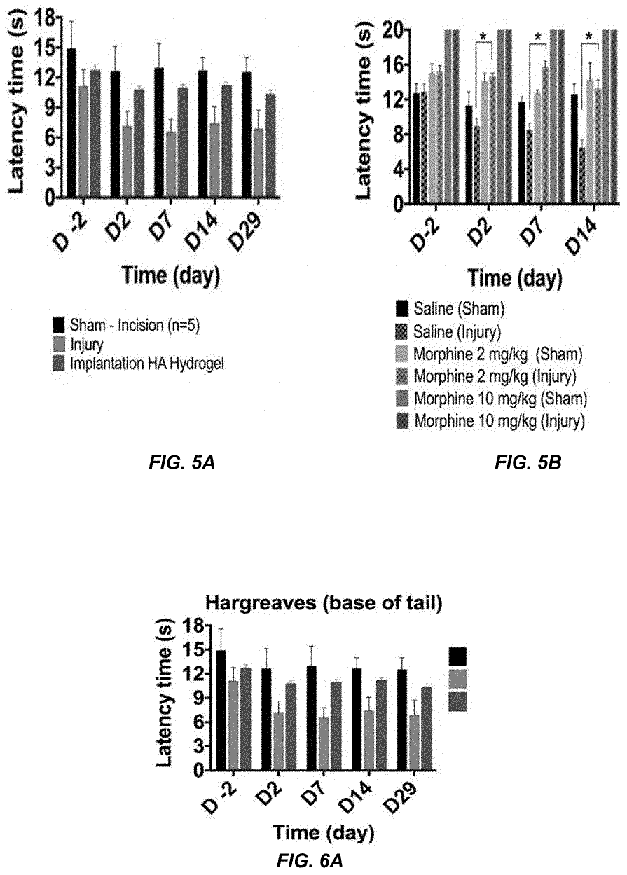

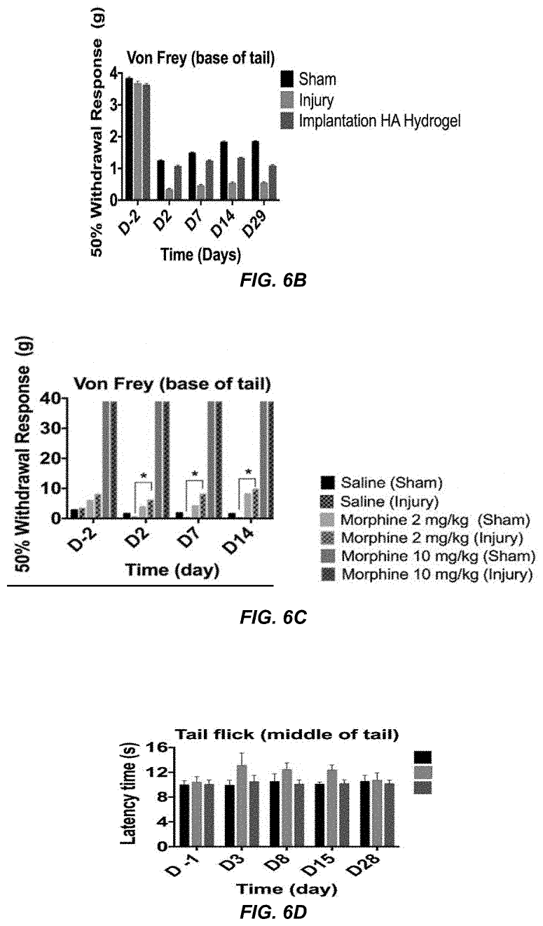

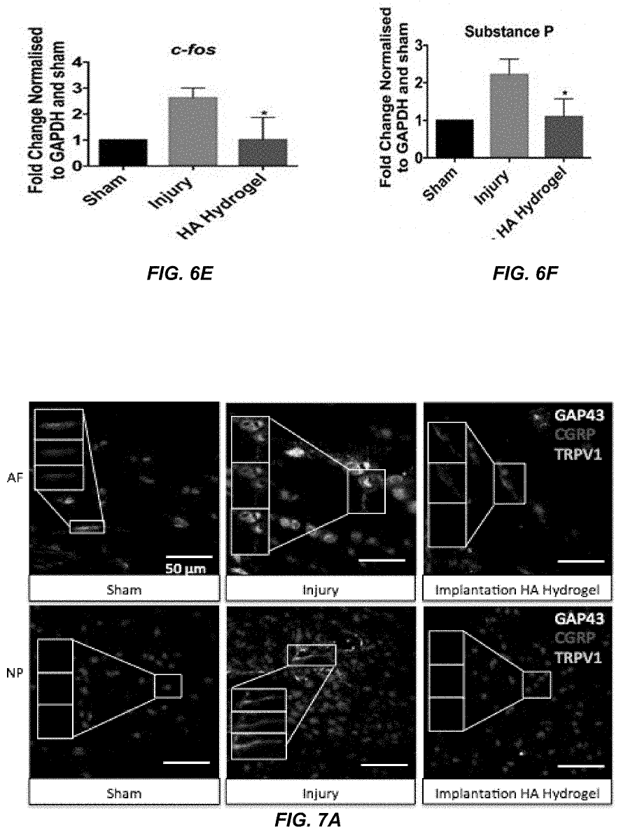

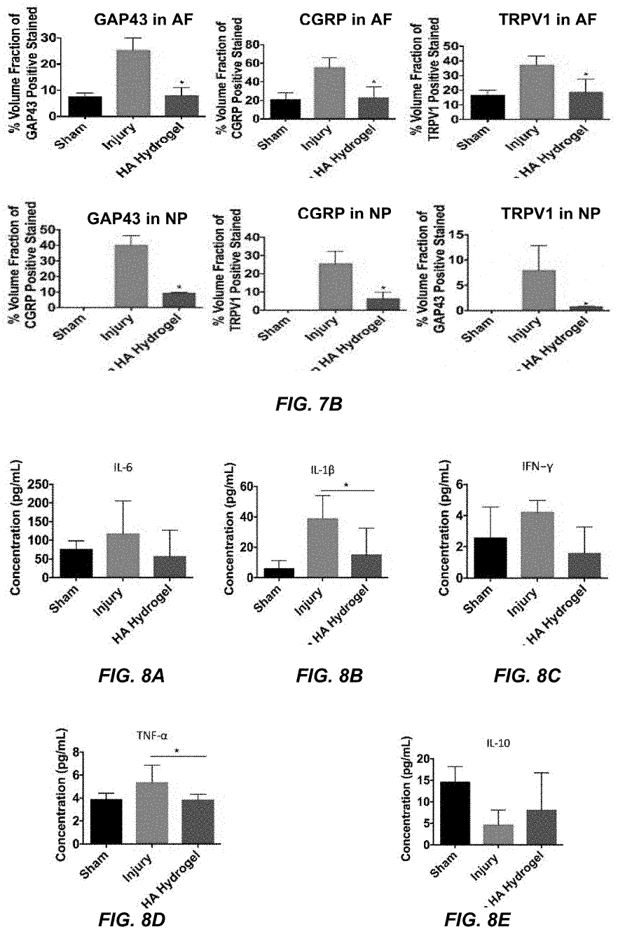

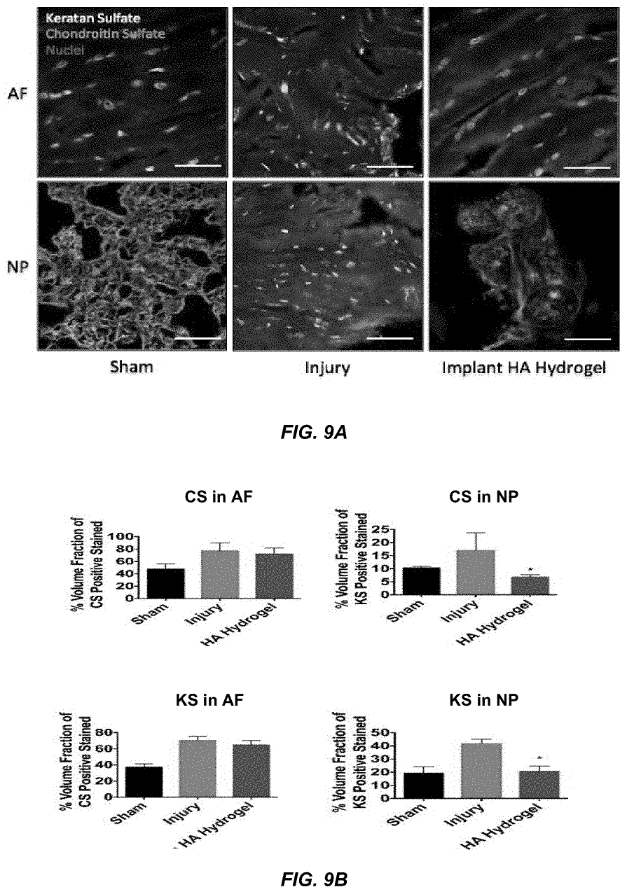

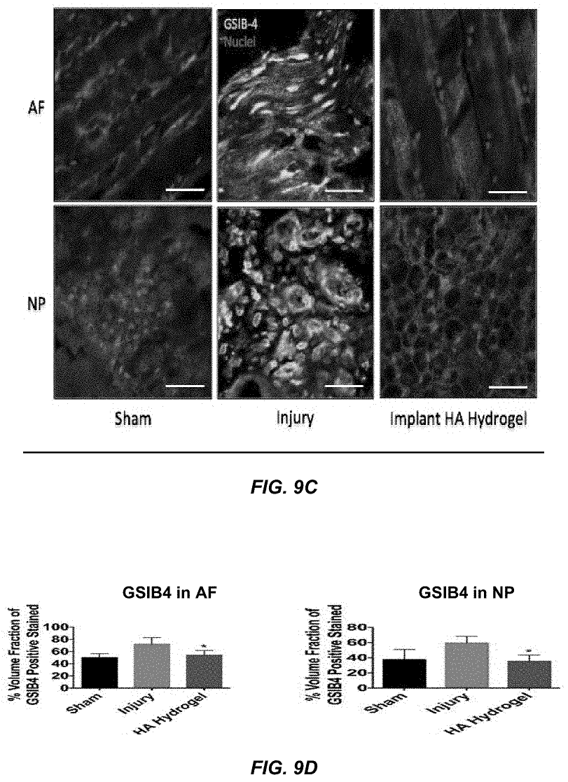

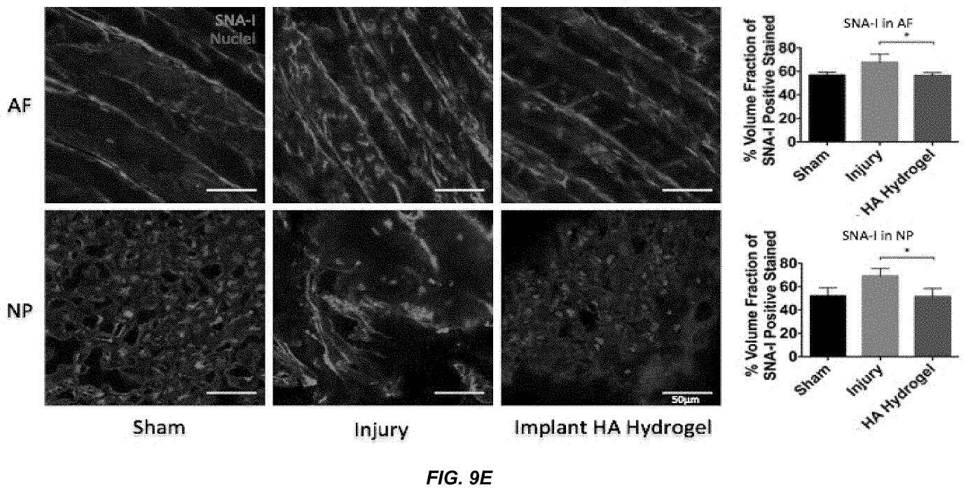

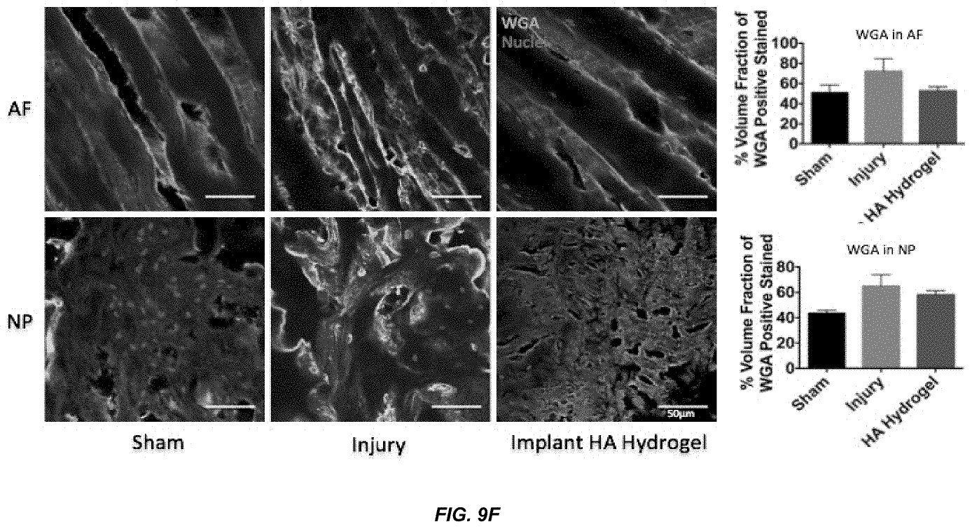

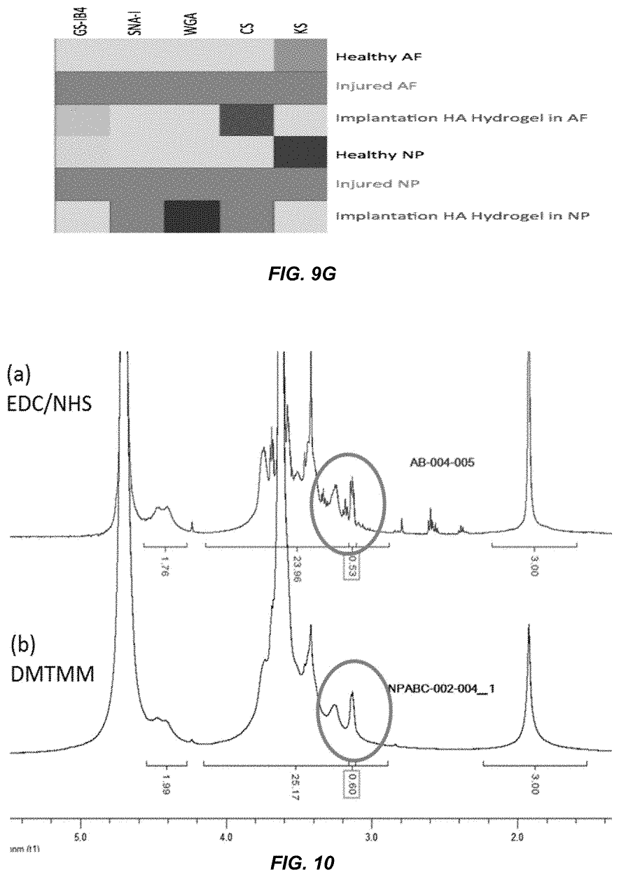

Rats were grouped into three groups: sham (n=20), injury (n=20) and injury with treatment implantable HA hydrogels (n=20). The hydrogel was implanted at the site of injury just after the injury was induced and the rats were assessed for pain behavior post-operative at days 1, 7, 14 and 28. The rats were then euthanized at day 7 and 29 to harvest the disc, spinal cord and blood plasma for analyses. Molecular pain marker of c-Fos and substance P were determined by qRT-PCR. Immunohistochemistry was used to identify reactivity to GAP43, CGRP protein and TRPV1 in the disc, an innervation, sensory neuropeptide and nociception receptor marker. Lectin histochemistry methods were adopted to analyse glycan expression in the disc. Circulatory cytokines of IL-1β, IL-6, IFN-Y, TNF-α and IL-10 in the blood plasma were measured by multiplex ELISA. A schematic of the study design is provided in FIG. 4. Implantation of the HA hydrogel displayed increased latency time in the Hargreaves test until day 29 and this suggests that the HA alleviates thermal hyperalgesia as comparable to morphine treatment as a positive control (FIG. 5A, FIG. 6A). A similar pattern was observed for the von Frey test, where the HA group showed an enhanced response compared to the injury group which indicates that the HA hydrogel reduced mechanical allodynia comparable with morphine (FIG. 6B and FIG. 6C). For the tail flick test, HA suppressed hypoalgesia by reducing the latency time to a similar value as that of the sham, which is comparable to low dose morphine. (FIG. 6D). At molecular level in central nervous system, gene expression analysis revealed HA attenuated injury-induced substance P (FIG. 6F and c-Fos (FIG. 6E) in the spinal cord. Immuno-staining of GAP43, CGRP and TRPV1 demonstrated HA inhibited injury-induced peripheral sensory innervation, sensory neuropeptide and pain receptor in AF and NP tissue. (FIGS. 7A and 7B). Circulating cytokines of IL-1β, IL-6, IFN-Y, TNF-α were decreased upon implantation of the HA hydrogel (FIGS. 8A-8D). This suggests that HA reduces systemic inflammation related to pain and this observation was supported by an increase of the anti-inflammatory marker IL-10. Chondroitin sulfate content was increased after implantation of the HA hydrogel in the disc. In contrast, keratan sulfate expression was decreased in response to implantation of the HA hydrogel (FIGS. 9A-9G).

### Example 5

DMTMM Crosslinking Data

It was found that the degree of cross-linking can be enhanced by using (4-(4,6-Dimethoxy-1,3,5-triazin-2-yl)-4-methylmorpholine) (DMTMM) instead of N-(3-Dimethylaminopropyl)-N′-ethylcarbodiimide; NHS: N-Hydroxysuccinimide (EDC/NHS) as it is not sensitive to changes in pH of the solution (FIG. 10). Broader signal at 3.15 ppm when using DMTMM (b) suggests a higher degree of cross-linking.

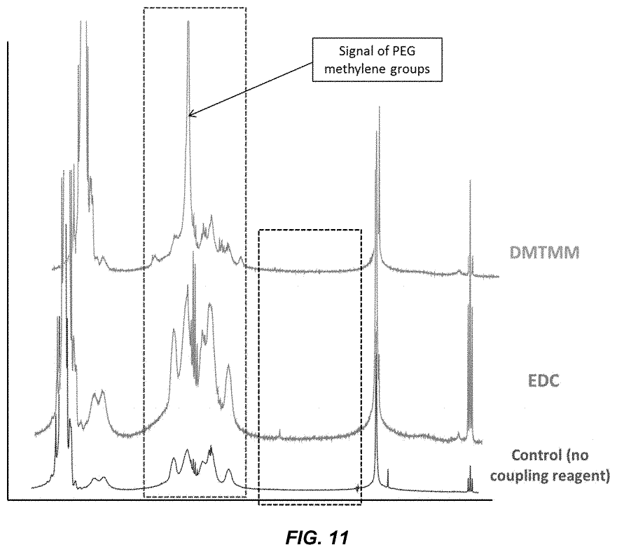

As FIG. 11 shows, it was confirmed that:
DMTMM was a more efficient reagent for cross-linking the polymer than EDC/NHS.
Purification method was also more efficient as the control reaction with no cross-linking reagent revealed only peaks for sodium hyaluronate in NMR spectra after purification.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A cross-linked high molecular weight hyaluronan (HA) hydrogel for use in a method of suppressing discogenic pain, in a mammal with intervertebral disc degeneration, in which the hydrogel is implanted in the mammal at a site of intervertebral disc degeneration.

2. A hydrogel of claim 1 for use of claim 1 in which the HA suppresses discogenic pain by the attenuation of sensory hyperinnervation.

3. A hydrogel of claim 1 for use of claim 1 or 2 in which the HA suppresses discogenic pain by reversal of thermal hyperalgesia or mechanical hyperalgesia.

4. A hydrogel of claim 1, for use of claim 1, in which in which the HA suppresses discogenic pain by the attenuation of nociception.

5. A hydrogel of claim 1, for use of any of claims 2 to 4, in which the hydrogel comprises a single gel network.

6. A hydrogel of claim 1 or 5, for use of any preceding Claim, in which the hydrogel comprises 0.1 to 10% cross-linked HA by weight.

7. A hydrogel of claim 1, 5 or 6, for use of any preceding Claim, in which the hydrogel comprises 0.5 to 2% cross-linked HA by weight.

8. A hydrogel of claim 1, 5 or 7, for use of any preceding Claim, in which the hydrogel is chemically crosslinked.

9. A hydrogel of claim 8, for use of any preceding Claim, in which the HA is cross-linked with PEG-amine.

10. A hydrogel of claim 8 or 9, for use of any preceding Claim, in which the HA crosslinking comprises EDC/NHS or DMTMM chemistry.

11. A hydrogel of any preceding Claim, for use of any preceding Claim, in which the HA is derivatised with a moiety that imparts a net positive charge on the HA.

12. A hydrogel of any preceding Claim, for use of any preceding Claim, in which the hydrogel additionally comprises a pharmaceutically or biologically active agent.

13. A hydrogel of any preceding Claim, for use of any preceding Claim, in which the pharmaceutically or biologically active agent is selected from the group consisting of: a cell, cell component, polysaccharide, protein, peptide, polypeptide, antigen, antibody (monoclonal or polyclonal), antibody fragment (for example an Fc region, a Fab region, a single domain antibody such as a nanobody or VHV fragment), a conjugate of an antibody (or antibody fragment) and a binding partner such as a protein or peptide, a nucleic acid (including genes, gene constructs, DNA sequence, RNA sequence, miRNA, shRNA, siRNA, anti-sense nucleic acid), a cellular product such as a growth factor (i.e. EGF, HGF, IGF-1, IGF-2, FGF, GDNF, TGF-alpha, TGF-beta, TNF-alpha, VEGF, PDGF and an interleukin) or a biological or non-biological drug.

14. A hydrogel of any preceding Claim, for use of any preceding Claim, and comprising high molecular weight hyaluronan particles dispersed throughout the high molecular weight hyaluronan hydrogel matrix.

15. A hydrogel of any preceding Claim, for use of any preceding Claim, in which the hydrogel is formulated for and administered by injection.

16. A hydrogel of any of claims 1 to 14, for use of any preceding Claim, in which the hydrogel is an implant and is surgically administered.

17. A cross-linked high molecular weight hyaluronan (HA) homopolymer hydrogel for use in a method of suppression of discogenic pain in a mammal with intervertebral disc degeneration, in which the hydrogel is implanted in the mammal at a site of intervertebral disc degeneration in which the HA suppresses discogenic pain by attenuation of hyperalgesia, hypoalgesia, sensory hyperinnervation and/or nociception.

18. A cross-linked high molecular weight hyaluronan (HA) hydrogel for use in a method of attenuation of hyperalgesia, hypoalgesia, sensory hyperinnervation and/or nociception in a mammal with intervertebral disc degeneration, in which the hydrogel is implanted in the mammal at a site of intervertebral disc degeneration.

19. A cross-linked high molecular weight hyaluronan (HA) hydrogel for use in a method of regeneration of an intervertebral disc in a mammal with intervertebral disc degeneration, in which the hydrogel is implanted in the mammal at a site of intervertebral disc degeneration.

20. A hydrogel of claim 19, for use of claim 19, in which the HA modulates endogenous extracellular matrix production to maintain or increase height.

21. A hydrogel of claim 19, for use of claim 19 or 20, in which the hydrogel comprises a single gel network.

22. A hydrogel of claim 19 or 21, for use of claim 19 or 20, in which the hydrogel comprises 0.1 to 10% cross-linked HA by weight.

23. A hydrogel of any of claims 19 to 22, for use of claim 19 or 20, in which the hydrogel comprises 0.5 to 2% cross-linked HA by weight.

24. A hydrogel of any of claims 19 to 23, for use of claim 19 or 20, in which the hydrogel is chemically crosslinked.

25. A hydrogel of any of claims 19 to 24, for use of claim 19 or 20, in which the HA is cross-linked with PEG-amine.

26. A hydrogel of any claims 24 to 25, for use of claim 19 or 20, in which the HA crosslinking comprises EDC/NHS or DMTMM chemistry.

27. A hydrogel of any claims 19 to 26, for use of claim 19 or 20, in which the HA is derivatised with a moiety that imparts a net positive charge on the HA.

28. A hydrogel of any claims 19 to 27, for use of claim 19 or 20, in which the hydrogel additionally comprises a pharmaceutically or biologically active agent.

29. A hydrogel of any claims 19 to 28, for use of claim 19 or 20, in which the pharmaceutically or biologically active agent is selected from the group consisting of: a cell, cell component, polysaccharide, protein, peptide, polypeptide, antigen, antibody (monoclonal or polyclonal), antibody fragment, a conjugate of an antibody or antibody fragment and a binding partner such as a protein or peptide, a nucleic acid, a cellular product such as a growth factor or a biological or non-biological drug.

30. A hydrogel of any claims 19 to 29, for use of claim 19 or 20, and comprising high molecular weight hyaluronan particles dispersed throughout the high molecular weight hyaluronan hydrogel matrix.

31. A hydrogel of any claims 19 to 30, for use of claim 19 or 20, in which the hydrogel is formulated for and administered by injection.

32. A hydrogel of any claims 19 to 30, for use of claim 19 or 20, in which the hydrogel is an implant and is surgically administered.

33. A cross-linked high molecular weight hyaluronan (HA) hydrogel for use in a method of regeneration of an intervertebral disc in a mammal with intervertebral disc degeneration, in which the hydrogel is implanted in the mammal at a site of intervertebral disc degeneration, in which the HA modulates endogenous extracellular matrix production to maintain or increase height.

34. A crosslinked high molecular weight hyaluronan (HA) hydrogel of any preceding Claim, for use of any preceding Claim, in which a non-crosslinked hydrogel and crosslinking agent are co-administered separately by injection and combined during or after administration to provide in-situ crosslinking of the hydrogel.
